(19) United States
(12) Patent Application Publication
(10) Pub. No.: US 2013/0323850 A1
(43) Pub. Date: Dec. 5, 2013

(54) OLIGONUCLEOTIDE IMMOBILIZED OSCILLATOR FOR DETECTING SILVER IONS AND METHOD OF DETECTING SILVER IONS USING RESONANCE FREQUENCY OF THE SAME

(71) Applicant: KOREA UNIVERSITY RESEARCH AND BUSINESS FOUNDATI, Seoul (KR)

(72) Inventors: Sung Soo Na, Seoul (KR); Jinsung Park, Seoul (KR); Kue-Whan Jang, Seoul (KR); Wook Choi, Daejeon (KR)

(73) Assignee: Korea University Research and Business Foundation, Seoul (KR)

(21) Appl. No.: 13/688,389

(22) Filed: Nov. 29, 2012

(30) Foreign Application Priority Data
May 30, 2012 (KR) 10-2012-0057409

(51) Int. Cl. G01N 33/20

(52) U.S. Cl. CPC G01N 33/20
USPC 436/80; 422/68.1

(57) ABSTRACT

The present disclosure relates to an oligonucleotide-immobilized microoscillator for detecting silver ions and a method for detecting silver ions using resonance of the same. A DNA including a thiol-terminated cytosine is immobilized on the surface of the microoscillator. In accordance with the present disclosure, silver nanoparticles detrimental to the human body and environment can be detected and quantified through a simple method based on the mechanical property of resonance frequency. Since trace silver nanoparticles below 1 nM can be detected with high sensitivity and selectivity, it may be usefully applied as a biosensor for detecting the toxic material.

### CROSS-REFERENCE TO RELATED APPLICATIONS

This application claims priority under 35 U.S.C. §119 to Korean Patent Application No. 10-2012-0057409 filed on May 30, 2012, in the Korean Intellectual Property Office, the disclosure of which is incorporated herein by reference in its entirety.

### TECHNICAL FIELD

The present disclosure relates to a method for detecting silver ions using resonance of an oligonucleotide-immobilized microoscillator for detecting silver ions. More particularly, the disclosure relates to a microoscillator capable of selectively detecting silver ions only with high sensitivity based on resonance frequency shift arising from mass increment occurring when silver ions are intercalated into a DNA, and a method for detecting silver ions using the same.

### BACKGROUND

With the recent rapidly increasing use of nanosized materials in scientific researches and industrial applications, negative effects of the nanosized materials on human health and environment are reported. Representative heavy metal nanomaterials widely used in scientific researches and industrial applications include copper, silver, aluminum, zinc, cadmium, etc.

In particular, the use of silver nanoparticles (AgNPs) has increased rapidly recently because of their antibacterial effect. They have been widely used in areas that can directly affect human health, including clothes, water purifiers, washing machines, toothpaste, and so forth. It is estimated that approximately 2,500 tons of silver is discharged to the environment as industrial wastes, 150 tons of which being discharged as sludge and 80 tons being released to surface water. The silver nanoparticles are easily ionized and diffuse into water, including sewage, river and tap water.

Silver ions (Ag⁺) are highly toxic to most bacteria, viruses, algae and fungi. They may inactivate sulfhydryl enzymes and accumulate in the human body. According to recent studies, those nanosized materials are reported to cause cell necrosis and severe diseases such as cancer, Parkinson's disease and Alzheimer's disease and have negative effects on the environment as well. Due to the danger of the AgNPs, researches on detection of AgNPs are actively carried out recently.

The existing methods for detecting the AgNPs include fluorescence-based detection using DNA and graphene and electrochemical detection using DNA and an electrode. They detect the AgNPs using electrical and chemical properties, not mechanical properties.

Currently, the interaction between metal ions and DNA base pairs attracts considerable attentions because of potential in sensing applications. Some metal ions are able to selectively bind to a native or synthetic DNA duplex to form metal-mediated base pairs. The thermal stability of the DNA duplex is increased due to the DNA-metal interaction.

Recently, the specific interaction of silver ions with cytosine-cytosine (C—C) mismatches has been focused on in development of Ag⁺ sensors. It was found out that Ag⁺ is able to bind specifically to two cytosines and promote the C—C mismatches to form stable base pairs [Ono, S. Cao, H. Togashi, M. Tashiro, T. Fujimoto, T. Machinami, S. Oda, Y. Miyake, I. Okamoto, Y. Tanaka, Chemical Communications, 2008]. They developed a fluorescence sensor for detecting silver ions using C—Ag⁺—C coordination chemistry.

Also, a graphene oxide-based sensor for detecting silver ions using a fluorophore-labeled oligonucleotide as a recognition unit and graphene oxide as a quencher was reported [Y. Wen, F. Xing, S. He, S. Song, L. Wang, Y. Long, D. Li, C. Fan, Chemical Communications, 2010, 46, 2596-2598].

Similarly, a single-walled carbon nanotube for detecting silver ions based on fluorescence spectroscopy was developed [C. Zhao, K. Qu, Y. Song, C. Xu, J. Ren, X. Qu, Chemistry: A European Journal, 2010, 16, 8147-8154].

Nonetheless, due to the high cost and unsatisfactory detection limit, it is desired to develop a system that is not only highly sensitive and reliable but also selective and economical in detection. In addition, development of a detection method using mechanical properties is necessary to overcome the limitation of the methods based on chemical or electrical properties.

### SUMMARY

The present disclosure is directed to providing a microoscillator capable of selectively detecting silver ions only with high sensitivity and a method for detecting silver using resonance of the microoscillator.

In one general aspect, there is provided a microoscillator for detecting silver ions wherein a DNA including a thiol-terminated cytosine is immobilized on the surface of the microoscillator.

In an exemplary embodiment of the present disclosure, the surface of the microoscillator on which the DNA is immobilized may be coated sequentially with chromium (Cr) and gold (Au) and the thiolated terminal of the DNA may be bound to the gold (Au).

In another exemplary embodiment of the present disclosure, the back side of the microoscillator on which the DNA is immobilized may be coated with aluminum.

In another exemplary embodiment of the present disclosure, the DNA may be 5′-(CCC)ₙ-HS-3′ (1≦n≦20).

In another general aspect, there is provided a method for detecting silver ions, including: (a) immobilizing a DNA including a thiol-terminated cytosine on the surface of a microoscillator; (b) reacting a sample containing silver ions with the surface of the microoscillator; and (c) measuring a resonance frequency shift of the microoscillator in real time.

In an exemplary embodiment of the present disclosure, the sample containing silver ions may further contain sodium nitrate and cytosine molecules.

In another exemplary embodiment of the present disclosure, the resonance frequency shift may be proportional to the mass of the cytosine molecules and captured silver ions and the resonance frequency shift may be a difference of a resonance frequency measured in real time from a reference resonance frequency.

In another exemplary embodiment of the present disclosure, wherein the reference resonance frequency may be a resonance frequency of the microoscillator measured in a sample not containing silver ions.

In another exemplary embodiment of the present disclosure, the method may further include, before the step (a), functionalizing the surface of the microoscillator. For example, the functionalizing may be achieved by coating the surface of the microoscillator sequentially with chromium (Cr) and gold (Au) and coating the back side of the microoscillator with aluminum.

In another exemplary embodiment of the present disclosure, the DNA may be 5′-(CCC)ₙ-HS-3′ (1≦n≦20).

In accordance with the present disclosure, the silver nanoparticles detrimental to the human body and environment can be detected and quantified through a simple method based on the mechanical property of resonance frequency. Since trace silver nanoparticles below 1 nM can be detected with high sensitivity and selectivity, it may be usefully applied as a biosensor for detecting the toxic material.

### BRIEF DESCRIPTION OF THE DRAWINGS

The above and other objects, features and advantages of the present disclosure will become apparent from the following description of certain exemplary embodiments given in conjunction with the accompanying drawings, in which:

FIG. 1 schematically illustrates a mechanism of silver ion detection using a silver-specific nucleotide-coated oscillator (SSNO) according to the present disclosure, showing a DNA immobilized on the surface thereof, single cytosine molecules acting as amplifiers and a resonance frequency shift occurring when silver ions are specifically intercalated between two cytosines;

FIG. 2 shows fluorescence microscopic images (a, b) and optical microscopic images (c, d) of an oligonucleotide comprising a cytosine immobilized on the surface of a microoscillator;

FIG. 3 shows tapping mode AFM images of the bare surface of an oscillator with no DNA bound (a), the surface of an oscillator on which a DNA is immobilized (b) and the surface of an oscillator on which a silver ion is bound to cytosine (c) as well as a grain size histogram of each state (d) (The size of the tapping mode images is 7 μm×7 μm);

FIG. 4 shows normalized resonance frequency shift of a silver-specific DNA-coated oscillator with respect to silver ion concentration (blue bars) (The gray bar is the result for a control experiment and the wine-colored line indicates the trend line for silver ion detection.);

FIG. 5 shows selectivity for silver ions (The concentration of silver ions (gray bar) and other competing ions (lithium, zinc, iron and sodium; blue bars) is 100 nM.);

FIG. 6 shows (a) resonance frequency of a silver-specific nucleotide-coated oscillator in tap water (green square) and 100 nM silver ions dissolved in tap water (blue square) and (b) normalized resonance frequency of the silver-specific nucleotide-coated oscillator for each state;

FIG. 7 shows resonance frequency shift and tapping mode AFM images of a silver-specific nucleotide-coated oscillator without (green box) or with (blue box) a cytosine molecule (i.e., amplifier) added (The inserted figure shows normalized resonance of Ag⁺-bound state without or with cytosine.);

FIG. 8 shows a result of (a) control experiment for physical adsorption without silver ions and (b) silver specificity control experiment for AT-15 DNA sequence; and

FIG. 9 shows normalized resonance frequency shift of a silver-specific DNA-coated oscillator as a function of the number of reuse.

### DETAILED DESCRIPTION OF EMBODIMENTS

Hereinafter, exemplary embodiments will be described in detail with reference to the accompanying drawings.

The present disclosure relates to a silver-specific nucleotide-coated oscillator (SSNO) and a novel method for detecting silver ions with high sensitivity using the same.

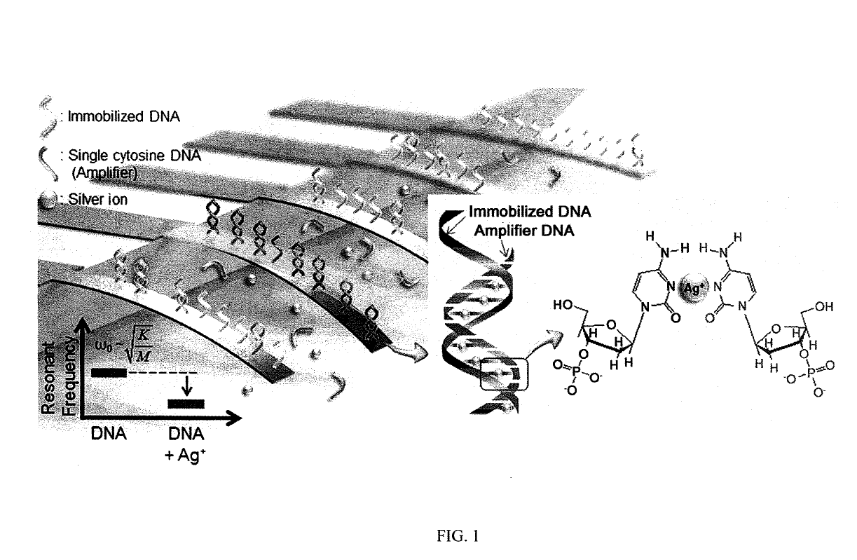

The basic design of the silver ion detection mechanism using the SSNO according to the present disclosure is displayed in FIG. 1.

In the method for detecting silver ions according to the present disclosure, for label-free detection of silver ions, the surface of the microoscillator is functionalized by immobilizing with a 30-base cytosine-rich oligonucleotide as an Ag₊-binding sequence. Specifically, a thiol-terminated cytosine-rich DNA is immobilized on the gold-coated microoscillator via covalent bonding.

That is to say, the surface of the microoscillator (SSNO) for detecting silver ions according to the present disclosure is functionalized with a DNA comprising a thiol-terminated cytosine so that a silver ion may be intercalated between two cytosines (cytosine-Ag⁺-cytosine).

To verify the immobilization of the cytosine-rich oligonucleotide on the surface of the microoscillator, a DNA comprising a fluorescein isothiocyanate (FITC)-tagged cytosine was used, which is thiolated for attachment to the gold (Au) surface of the oscillator.

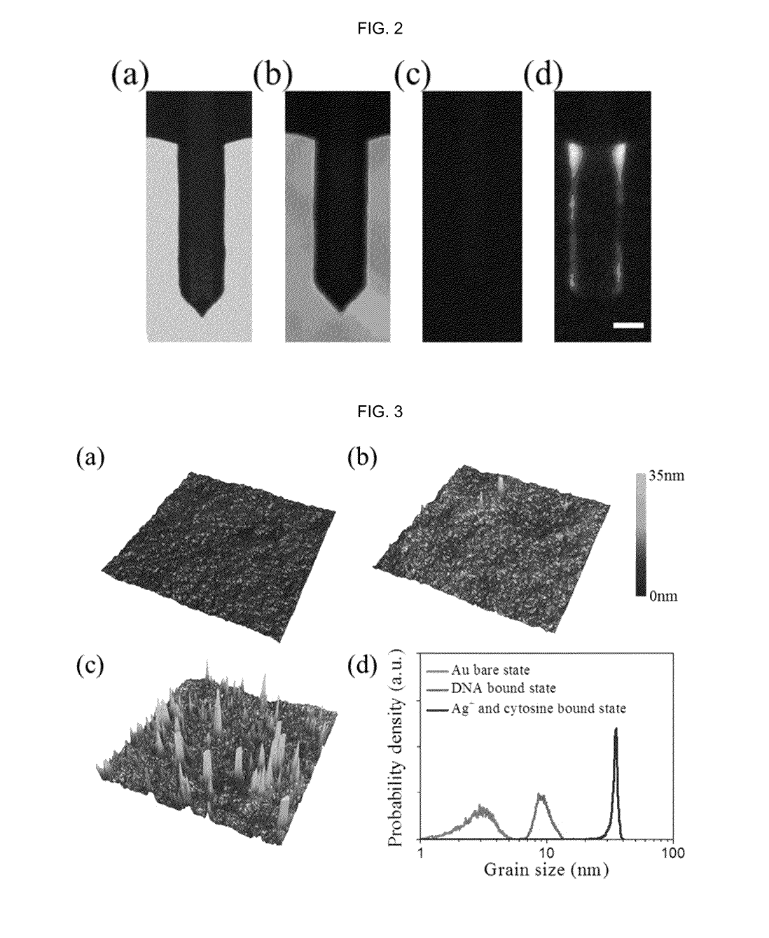

As shown in FIG. 2 (a) and (b), the gold-coated oscillator and the gold-coated oscillator with the FITC-tagged DNA show similar morphologies when observed under an optical microscope. However, as shown in FIG. 2 (c) and (d), the fluorescence microscopic images verify the oligonucleotide immobilized on the surface of the microoscillator.

The surface of the microoscillator may be coated sequentially with chromium (Cr) and gold (Au) and the thiolated terminal of the DNA may be bound to the gold (Au). And, the back side of the microoscillator may be coated with aluminum to enhance resonance accuracy owing to increased laser reflection.

The thiol-terminated silver-specific nucleotide may be 5′-(CCC)ₙ-HS-3′ (1≦n≦20). As a specific example, it may be 5′-CCC CCC CCC CCC CCC CCC CCC CCC CCC CCC-HS-3′.

The method for detecting silver ions in real time using resonance of the silver-specific nucleotide-coated oscillator according to the present disclosure comprises:

(a) immobilizing a DNA including a thiol-terminated cytosine on the surface of a microoscillator;

(b) reacting a sample containing silver ions with the surface of the microoscillator; and

(c) measuring a resonance frequency shift of the microoscillator in real time. The sample containing silver ions may further contain sodium nitrate to improve stability of the DNA duplex and may further contain single cytosine molecules to improve silver ion detection efficiency.

For sensitive detection of silver ions, mass amplification and nucleotide stability are required.

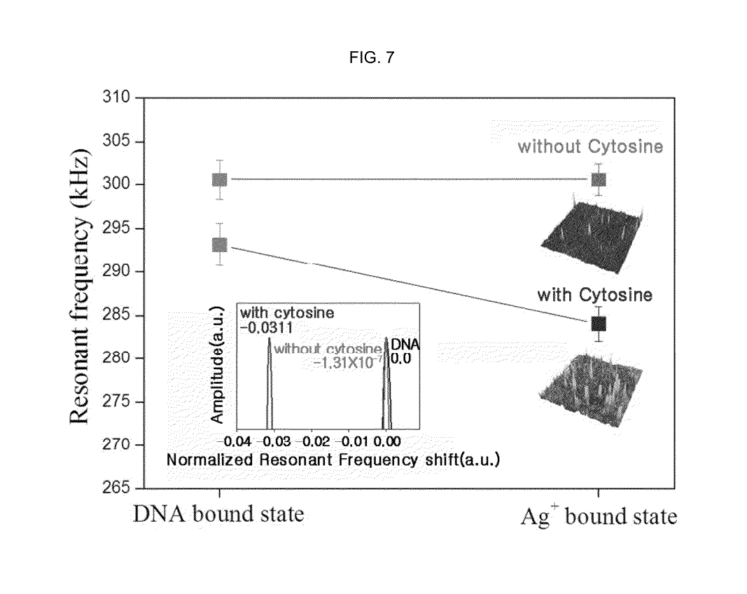

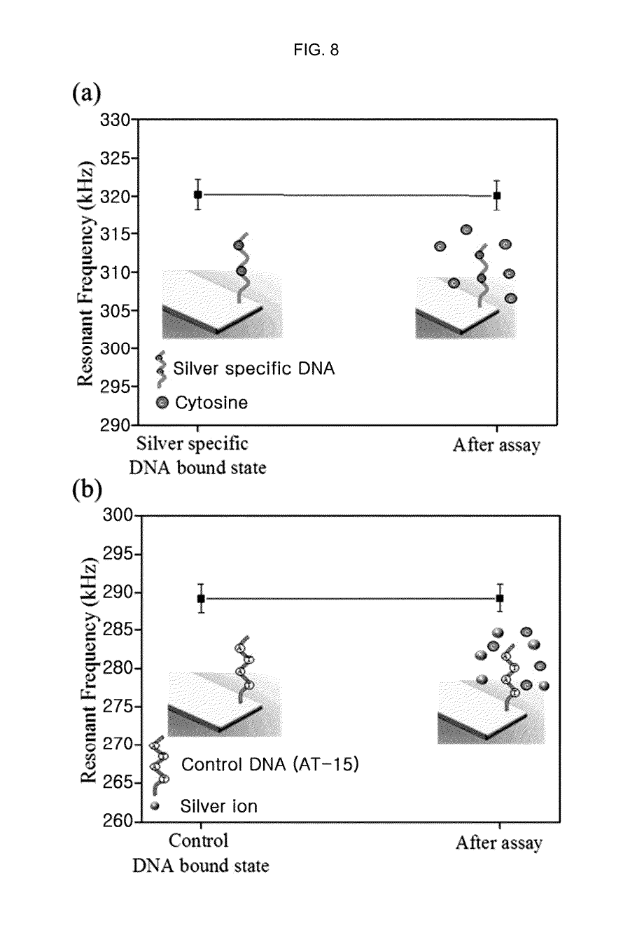

As shown in FIG. 7, the single cytosine molecules play the role of a mass amplifier which is able to form a C—Ag⁺—C base pair and widen the resonance frequency shift. Furthermore, the C—Ag⁺—C base pair increase the nucleotide stability. And, as shown in FIG. 8 (a), the single cytosine molecule serves as a maximally appropriate amplifier due to the cytosine-cytosine repulsion. Since the immobilized single-stranded cytosine-rich DNA is hydrophobic, water molecules allow the nucleotide to be a one-dimensional structure, which enables silver ions and cytosine molecules to easily access the immobilized DNA. And, as shown in FIG. 8 (b), the cytosine-rich DNA according to the present disclosure is able to specifically detect silver ions when compared with other DNA sequences such as adenine or thymine.

The detection method according to the present disclosure allows detection of silver ions based on the mechanical property of resonance frequency shift occurring after reaction with the sample. The resonance frequency (ω₀=(K/m)^{1/2}) shift is measured as a change in resonance frequency due to the mass change occurring when the silver ion is intercalated into the DNA. In the present disclosure, the single cytosine molecule is further included as a mass amplifier to improve the silver ion detection efficiency. In this case, the resonance frequency shift is proportional to the mass of the cytosine molecule and the captured silver ion.

The principle of silver ion detection based on the resonance frequency shift of the microoscillator according to the present disclosure is as follows.

The SSNO-based silver ion detection system according to the present disclosure detects silver ions based on the resonance frequency shift due to mass change.

In a dry air state, a rectangular oscillator exhibits the resonance behavior of the classical elastic continuum model. It is well known that resonance frequency shift of the microoscillator is attributed to the mass increase and/or decrease owing to molecule interaction such as molecular absorption and/or proteolysis.

Since the thickness of the oscillator according to the present disclosure is much larger than that of the molecular layer, the mass effect is a dominant factor for resonance behavior.

The resonance behavior of the microoscillator follows the classical elastic continuum model. In a dry air state, it may be described by Equation 1:

$${ω}_{0} = {( \frac{α}{L} )}^{2} \sqrt{\frac{EI}{{ρ}_{c} A}} = \sqrt{\frac{{K}_{c}}{{M}_{c}}} \qquad ( 1 )$$

In Equation 1, El, A, L and ρ_{c} are the oscillator's bending rigidity, cross-sectional area, length and density, respectively. The α is a solution of a transcendental equation such as cos α coshα+1=0. A representative solution of the transcendental equation is α₁=1.875 and α₂=4.694. And, the M_{c} and K_{c} are effective mass and effective stiffness of the oscillator, respectively. More specifically, they are given by M_{c}=ρ_{c}A and K_{c}=α⁴El/L⁴.

From this equation, the resonance frequency of the microoscillator according to the present disclosure is predicted as 366.7 kHz. The average resonance frequency is experimentally measured as 334.7±16.3 kHz. Here, the dimension of the microoscillator (cantilever) according to the present disclosure is L×ω^{c}×t_{c} (length×width×thickness), where L=125 μm, ω_{c}=40 μm and t_{c}=4 μm.

It means that the oscillator according to the present disclosure satisfies the classical elastic continuum model. Since the thickness of the microoscillator is much larger than the thickness of the molecular layer, the mass is the dominant cause of the resonance frequency shift.

That is to say, the silver ion and the single cytosine molecule play the role of mass increment in the present disclosure. From Equation 2, it can be seen that the resonance frequency shift is directly related to the molecular mass.

$$\frac{{Δω}_{0}}{{ω}_{0}} = ( \frac{1}{2} ) ( \frac{Δ M}{{M}_{c}} ) \qquad ( 2 )$$

where Δω₀ is the resonance frequency shift measured in dry air and ΔM is the total mass of molecules including molecule interaction. Based on this resonance frequency shift, the SSNO according to the present disclosure is able to detect the target silver ions.

### EXAMPLES

The examples and experiments will now be described. The following examples and experiments are for illustrative purposes only and not intended to limit the scope of this disclosure.

### Fabrication Example 1

### Fabrication of Silver-Specific Nucleotide-Coated Oscillator (SSNO)

(1) A microoscillator with a force constant of about 42 N/m (TESPA, Bruker, Calif.) whose dimension is 40×41×25 μm³ (width×thickness×length) was used. The back side of the oscillator was coated with aluminum to enhance resonance accuracy owing to laser reflection. The resonance frequency of the oscillator was in the range of about 300-365 kHz in dry air.

Chromium (Cr) and gold (Au) layers (100 Å and 200 Å, respectively) were deposited on the microoscillator using an electron-beam evaporator (Maestech, Korea) to immobilize an oligonucleotide comprising cytosine (C-rich oligonucleotide). Thereafter, the microoscillator was washed with ethanol and triple distilled water (pH 7.5; Millipore, Bedford, Mass.) for several times and dried in vacuum for 12 hours at room temperature for complete drying.

(2) The sequence of the thiol-terminated silver-specific nucleotide was 5′-CCC CCC CCC CCC CCC CCC CCC CCC CCC CCC-HS-3′ (Integrated DNA Technology, CA, USA). The nucleotide was dissolved in aqueous Tris-EDTA buffer (pH 8). In order to immobilize the nucleotide on the oscillator, the microoscillator was immersed in a DNA buffer (50 μL, 10 μM) for 2 hours. The thiol-modified DNA was then attached on the gold surface of the oscillator.

After gently washing the oscillator with deionized water, the oscillator was dried in vacuum for 12 hours at room temperature for perfect drying. Following the drying, a silver-specific nucleotides coated oscillator (SSNO) was obtained.

### Fabrication Example 2

### Fabrication of SSNO

An SSNO was fabricated in the same manner as in Fabrication Example 1 except that a fluorescein isothiocyanate (FITC)-tagged DNA was immobilized on the microoscillator.

The sequence of the DNA was the same as in Fabrication Example 1 except for the fluorescent dye (5′-56-FAM-CCC CCC CCC CCC CCC CCC CCC CCC CCC CCC-HS-3′).

### Example 1

### Analysis of Tapping Mode Atomic Force Microscopic (AFM) and Scanning Electron Microscopic (SEM) Images

Atomic force microscopy (AFM) can be operated in a number of modes, including contact mode, tapping mode, phase image mode, etc. In this example, tapping mode AFM was used to detect the resonance frequency of the microoscillator (cantilever).

Tapping mode AFM measurements were performed using an Innova microscope (Veeco Corp., Santa Barbara, Calif., USA) with a nanodrive controller (Veeco, Santa Barbara, Calif., USA) in air at ambient temperature and pressure.

A closed-loop scanner was used to obtain precise and reproducible tapping mode image. A TESPA cantilever tip was used for recording all images. The resonance frequency of this cantilever was 320 kHz and the tip radius was about 10 nm.

All the images' sizes were 700 nm×700 nm with scanning at 1 Hz. All images were leveled in two dimensions and processed with the SPM Lab Analysis software V7.00 (Veeco Corp., Santa Barbara, Calif., USA). Probability analysis was performed with the Nanoscope analysis software V1.20 (Bruker Corp., Santa Barbara, Calif., USA). Also, morphology was observed by scanning electron microscopy (SEM) using the Hitachi model S-5300 microscope.

To detect silver ions using the SSNO, the tapping mode AFM was used to obtain clear molecular size distribution because the contact mode ATM is not suitable for biomolecular samples because of drag force that may aggravate the sample.

As shown in FIG. 3, the tapping mode AFM provides the grain size distribution of the surface morphology on the gold (Au) surface of the microoscillator. FIGS. 3 (b) and (c) depict the change of the surface morphology driven by the C-rich oligonucleotide (10 μM) binding onto the gold surface and adsorption of Ag⁺ (100 nM) by single cytosine molecules (10 μM). FIG. 3 (d) shows the grain size diagram of bare Au state, silver-specific DNA-bound state and silver ion- and single cytosine molecule-bound state, respectively.

The grain size distribution follows the normal distribution: f(x)=A exp[−(x−μ)²/(2σ²)], where f(x) is the probability function for grain size x, μ is the mean value of the grain size, and σ² is the variance.

As shown in FIG. 3 (a), the bare gold surface exhibits a small grain size (3.07±0.8 nm, μ±σ²) whereas the surface with the C-rich DNA immobilized possesses an increased grain size of 9.32±1.2 nm (FIG. 3 (b)). It implies that the silver-specific nucleotide was well attached to the gold (Au) substrate. After binding of Ag⁺ and the cytosine amplifier to the DNA-immobilized oscillator, the largest grain size 34.93±1.29 nm was observed (FIG. 3 (c)). From this result, it was confirmed that the single cytosine molecules play the critical role of mass amplifier and enhance the silver ion capturing and detecting ability of the SSNO-based sensing system.

### Example 2

### Ag⁺ Detection and Selectivity Assay

Silver ions were prepared by dissolving silver nitrate (AgNO₃) in deionized water. The nucleotide-immobilized microoscillator was immersed in the silver ion solution (200 μL) with single cytosine molecules (200 μL, 100 μM) as a mass amplifier. Also, NaNO₃ (100 μL, 50 mM) was added to the solution to increase the stability of the DNA. After incubation for 2 hours, the SSNO was dried for 12 hours in vacuum state for perfect drying. Four metal salt solutions of sodium nitrate (Sigma-Aldrich), lithium nitrate (Sigma-Aldrich), zinc nitrate hexahydrate and iron (III) chloride (Sigma-Aldrich) were prepared to investigate the detection selectivity of the SSNO. The concentration of all the metal ions was 100 nM that was the same as that of silver ions.

### Example 3

### General Tap Water Experiment

General tap water was used to investigate the detection possibility of silver ions in the same manner as in Example 2. The experimental solution was leached from normal tap water by using a syringe filter with a pore size of 0.2 μm (Minisart, Sigma-Aldrich). Silver nitrate (100 nM), single cytosine molecules (100 μM) and sodium nitrate (50 mM) were dissolved in the filtered tap water.

### Example 4

### Control Experiment for Effect of Cytosine, Physical Adsorption and Reactivity of Other DNA

(1) To investigate the effect of the single cytosine molecules, the silver-specific DNA-immobilized oscillator was added into two different solutions.

One solution contained silver nitrate (10 μM) and sodium nitrate (50 mM) and the other solution contained silver nitrate (10 μM), sodium nitrate (50 mM) and cytosine molecules (100 μM). As seen from FIG. 7, the single cytosine molecules played the role of enhancing the resonance frequency of the SSNO.

(2) To investigate the possibility of physical absorption, the same SSNO was prepared and added into an assay solution containing single cytosine molecules (100 μM) and sodium nitrate (50 mM). As seen from FIG. 8 (a), there was no difference in resonance frequency between before and after the assay.

(3) To investigate the silver specificity of the SSNO, the DNA sequence of the SSNO fabricated in Fabrication Example 1 was changed with adenine and thymine. The sequence of the control DNA (AT-15) was 5′-ATA TAT ATA TAT ATA TAT ATA TAT ATA TAT-HS-3′, with the same length and concentration (10 μM).

The AT-15-immobilized oscillator was immersed into a solution (containing silver ions, sodium nitrate and cytosine molecules) of the same concentration as that of Example 2. As seen from FIG. 8 (b), there was no difference in resonance frequency after the assay. This confirms that the DNA according to the present disclosure is a silver-specific DNA.

To investigate the silver ion capturing ability of the SSNO according to the present disclosure, experiments were performed for different Ag⁺ concentrations. First, to determine the capturing sensitivity, the resonance frequency of the oscillator owing to the disparity of initial resonance of each cantilever was normalized.

The normalized resonance frequency is described as ωₛ=(ω_{d}−ωᵢ)/ω_{b}×100, where ω_{d} and ωᵢ are the resonance frequency of the silver-specific DNA-bound state and silver ion- and cytosine molecule-bound state, respectively, and ω_{b} is the resonance frequency of a non-treated oscillator.

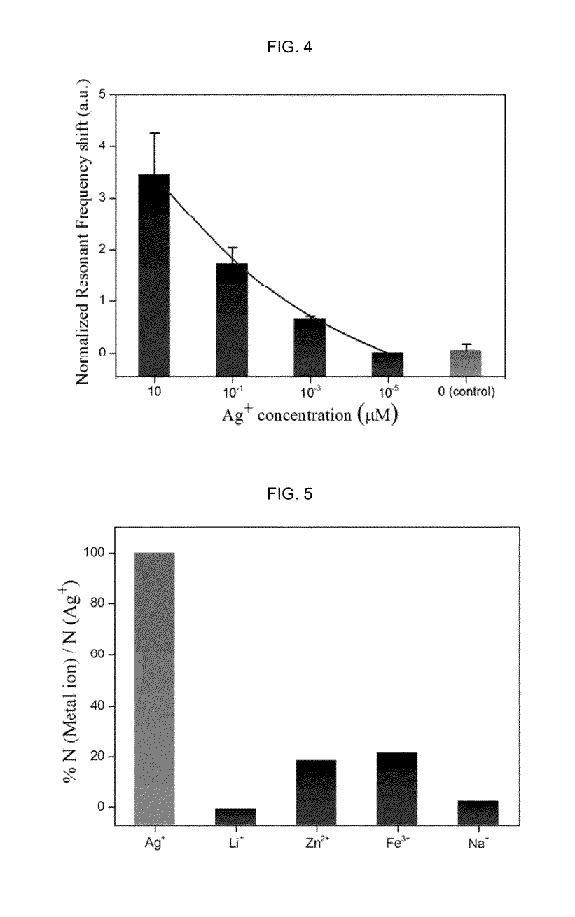

As shown in FIG. 4, the normalized resonance frequency shift of the SSNO shows a high sensitive tendency toward Ag⁺ concentrations. A downward trend was obtained between the normalized resonance frequency shift and the concentration of silver ions from 10 pM to 10 μM. The decrement of the standard deviation (from 10 μM to 10 pM) indicates that a reaction between the SSNO and the target ion occurs actively at high concentration.

The limit of detection (LOD) of the SSNO was below 1 nM (0.642±0.072, ωₛ±standard deviation), which is 10-fold larger than the resonant shift for the control sample (0 nM, 0.045±0.127). More specifically, the detection limit of the SSNO sensing mechanism was between 1 nM and 10 pM, which is more sensitive than those of the previous reports using fluorescence probes (10 nM) [A. Ono, S. Cao, H. Togashi, M. Tashiro, T. Fujimoto, T. Machinami, S. Oda, Y. Miyake, I. Okamoto, Y. Tanaka, Chemical Communications, 2008], graphene-based probes (20 nM) [Y. Wen, F. Xing, S. He, S. Song, L. Wang, Y. Long, D. Li, C. Fan, Chemical Communications, 2010, 46, 2596-2598] and metal nanoparticles (5 nM) [G. D. Huy, M. Zhang, P. Zuo, B.-C. Ye, Analyst, 2011, 136, 3289-3294].

To confirm that Ag⁺ can be detected selectively with the SSNO according to the present disclosure, the same experiment was performed for four representative metal ions Li⁺, Zn²⁺, Fe³⁺ and Na⁺. The four metal ions are representative monovalent, bivalent or trivalent ions.

As seen from FIG. 5, selectivity for Ag⁺ (green bar) was superior to those for other metal ions (blue bars), although some other metal ions such as Zn²⁺ and Fe³⁺ showed reactivity with the SSNO.

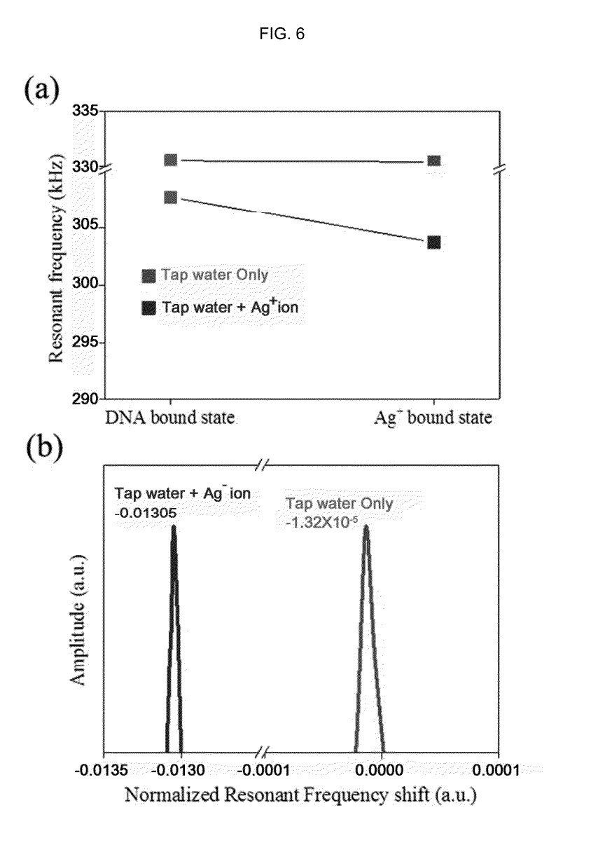

In addition, silver ion detection was performed using the SSNO according to the present disclosure in real tap water. FIG. 6 compares the resonance frequency of the C-rich nucleotide-bound state and the silver ion-bound state in blank tap water (green square, (a)) and in tap water in which Ag⁺ (100 nM) is dissolved (blue square, (a)).

Resonance frequency decrement occurred slightly (˜0.13 kHz) in the real tap water, which was below the error range. On the other hand, in the silver ion-dissolved tap water, the resonance frequency decreased distinctly (˜4.05 kHz) (FIG. 6 (b)). Accordingly, it can be seen that the SSNO according to the present disclosure is capable of selectively detecting Ag⁺ when it exists together with other interfering metal ions (Ca²⁺, Mg²⁺, Na⁺ and K⁺).

### Example 5

### Reusability of SSNO

In order to reuse the SSNO, organic matter was removed from the SSNO by immersing in piranha solution (1:1 mixture of H₂SO₄ and H₂O₂). The SSNO was then washed with triple distilled water and dried in vacuum at room temperature for 12 hours. The resonance frequency of the dried oscillator revealed that the surface was bare gold (Au) surface. Thereafter, the oscillator was immersed for 2 hours in aqueous Tris-EDTA buffer (50 μL, 10 μM, pH 8) in which a thiol-terminated silver-specific nucleotide is dissolved to immobilize the C-rich oligonucleotide. After washing with deionized water and drying in vacuum at room temperature for 12 hours, a fresh SSNO was obtained.

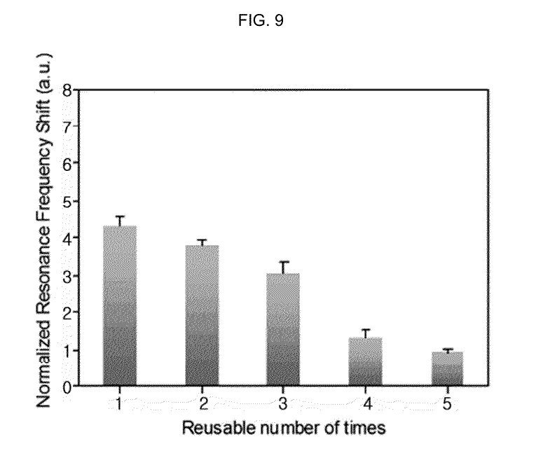

FIG. 9 shows the normalized resonance frequency shift of the silver-specific DNA-coated oscillator as a function of the number of reuse. The normalized resonance frequency shift is described as ωᵣ=(ωₐ−ω_{d})/ω_{b}×100, where ωₐ and ω_{d} are the resonance frequency of the gold-coated state and the silver-specific DNA-bound state, respectively, and ω_{b} is the resonance frequency of a non-treated oscillator.

As described above, the silver-specific nucleotide-coated oscillator (SSNO) according to the present disclosure is capable of selectively detecting the nanotoxic silver ions based on the sandwich method.

The effectiveness of the SSNO was confirmed through surface analysis by tapping mode AFM. It was shown that the resonant oscillator enables to capture Ag⁺ below 1 nM, which is the lowest concentration when compared with existing detection methods. Also, the SSNO-based method showed selectivity for Ag⁺ over other interference ions even in real tap water.

While the present disclosure has been described with respect to the specific embodiments, it will be apparent to those skilled in the art that various changes and modifications may be made without departing from the spirit and scope of the disclosure as defined in the following claims.

What is claimed is:

1. A microoscillator for detecting silver ions wherein a DNA comprising a thiol-terminated cytosine is immobilized on the surface of the microoscillator.

2. The microoscillator for detecting silver ions according to claim 1, wherein the surface of the microoscillator on which the DNA is immobilized is coated sequentially with chromium (Cr) and gold (Au) and the thiolated terminal of the DNA is bound to the gold (Au).

3. The microoscillator for detecting silver ions according to claim 1, wherein the back side of the microoscillator on which the DNA is immobilized is coated with aluminum.

4. The microoscillator for detecting silver ions according to claim 1, wherein the DNA is 5′-(CCC)ₙ-HS-3′ (1≦n≦20).

5. A method for detecting silver ions, comprising:
immobilizing a DNA including a thiol-terminated cytosine on the surface of a microoscillator;
reacting a sample containing silver ions with the surface of the microoscillator; and
measuring a resonance frequency shift of the microoscillator in real time.

6. The method for detecting silver ions according to claim 5, wherein the sample containing silver ions further contains sodium nitrate and cytosine molecules.

7. The method for detecting silver ions according to claim 5, wherein the resonance frequency shift is proportional to the mass of the cytosine molecules and captured silver ions and the resonance frequency shift is a difference of a resonance frequency measured in real time from a reference resonance frequency.

8. The method for detecting silver ions according to claim 7, wherein the reference resonance frequency is a resonance frequency of the microoscillator measured in a sample not containing silver ions.

9. The method for detecting silver ions according to claim 5, which further comprises, before said immobilizing the DNA on the surface of the microoscillator, functionalizing the surface of the microoscillator.

10. The method for detecting silver ions according to claim 9, wherein said functionalizing comprises coating the surface of the microoscillator sequentially with chromium (Cr) and gold (Au) and coating the back side of the microoscillator with aluminum.

11. The method for detecting silver ions according to claim 5, wherein the DNA is 5′-(CCC)ₙ-HS-3′ (1≦n≦20).
